(19) United States
(12) Patent Application Publication
(10) Pub. No.: US 2015/0366839 A1
(43) Pub. Date: Dec. 24, 2015

(54) Schisantherin A for the treatment of neurodegenerative diseases

(71) Applicant: University of Macau, Macau (CN)

(72) Inventors: Ming Yuen LEE, Macau (CN); Fei SA, Macau (CN)

(21) Appl. No.: 14/742,703

(22) Filed: Jun. 17, 2015

Related U.S. Application Data
(60) Provisional application No. 62/013,553, filed on Jun. 18, 2014.

(51) Int. Cl. A61K 31/36

(52) U.S. Cl. CPC A61K 31/36

(57) ABSTRACT

This invention is directed to the use of Schisantherin A or Schizandrin C in treating and/preventing neurodegenerative diseases.

### CROSS-REFERENCE TO RELATED APPLICATION

This application claims benefit under 35 U.S.C. §119(e) of U.S. Provisional Application having Ser. No. 62/013,553 filed on 18 Jun. 2014, which is hereby incorporated by reference herein in its entirety.

### FIELD OF INVENTION

This invention relates to a dibenzocyclooctadiene lignan and in particular its use for treating neurodegenerative diseases.

### BACKGROUND OF INVENTION

Parkinson's disease (PD), as a slowly progressive neurodegenerative disease affecting 4-6 million people worldwide, is the second most common neurodegenerative disease caused by the loss of dopaminergic (DA) neurons in the substantia nigra pars compacta (SNpc), characterized by debilitating symptoms such as resting tremor, rigidity and bradykinesia (Rodriguez-Pallares, Parga et al. 2007). Although dopamine replacement method currently used for PD therapy improves clinical symptoms, the method does not retard the progression of the disease. So far, very few pharmacological agents have been isolated or developed that effectively inhibit the progression of PD. Therefore, new drugs with better curative effects and fewer side effects for PD therapy are urgently desired.

### SUMMARY OF INVENTION

In the light of the foregoing background, it is an objective of the present invention to provide an alternate therapeutic candidate for treating and/or preventing neurodegenerative diseases.

Accordingly, in one aspect, the present invention provides a method of treating and/or preventing neurodegenerative disease comprising administrating a pharmaceutically effective amount of a compound to a subject in need thereof. The compound is Schisantherin A, Schizandrin C or a combination thereof.

In an exemplary embodiment of the present invention, the neurodegenerative disease is caused by dopaminergic neuron loss or Aβ1-42 induced memory impairment.

In another exemplary embodiment of the present invention, the neurodegenerative disease is selected from the group consisting of Parkinson's disease and Alzheimer's disease.

In another aspect, the present invention provides a pharmaceutical composition comprising a pharmaceutically effective amount of a compound and a pharmaceutically acceptable carrier. The compound is Schisantherin A, Schizandrin C or a combination thereof.

In an exemplary embodiment of the present invention, the pharmaceutical composition can be prepared as microemulsion, tablets, granules, injection, powder, solution, suspension, sprays, patches or capsules.

In a further aspect, the present invention provides a method of treating and/or preventing neurodegenerative disease comprising administrating the pharmaceutical composition to a subject in need thereof.

### BRIEF DESCRIPTION OF FIGURES

FIG. 1 shows the chemical structures of the five lignans of S. chinensis.

FIG. 2A shows the cell viability of SH-SY5Y cells with different concentrations of Schisantherin A (SchiA). FIG. 2B shows the cell viability of SH-SY5Y cells pretreated with different concentrations of SchiA and then exposed to 6-OHDA. FIG. 2C shows the cell viability of SH-SY5Y cells pretreated with different concentrations of SC and then exposed to 6-OHDA. Values represent mean±S.E.M of 3 independent experiments. ###P<0.005 versus control group; *p<0.05, **p<0.01 and *** p<0.005 versus 6-OHDA group.

FIG. 3 shows the effect of SchiA in attenuating 6-OHDA-induced accumulation of ROS in SH-SY5Y cells.

FIG. 4A shows the effect of SchiA on the 6-OHDA-induced NO production in SH-SY5Y cells quantified by a multilabel counter. FIG. 4B shows the effect of SchiA on the 6-OHDA-induced iNOS expression in SH-SY5Y cells by western blot analysis. FIG. 4C shows the effect of SchiA on the 6-OHDA-induced iNOS expression in SH-SY5Y cells by densitometric analysis.

FIG. 5A shows the effect of SchiA on the levels of ERK phosphorylation in SH-SY5Y cells exposed to 6-OHDA by western blot analysis. FIG. 5B shows the effect of SchiA on the levels of ERK phosphorylation in SH-SY5Y cells exposed to 6-OHDA by densitometric analysis.

FIG. 6A shows the Akt signaling pathways involved in the neuroprotective action of SchiA on 6-OHDA-induced SH-SY5Y cell damage by western blot analysis. FIG. 6B shows the Akt signalling pathways involved in the neuroprotective action of SchiA on 6-OHDA-induced SH-SY5Y cell damage by densitometric analysis.

FIG. 7A shows the prevention of SchiA on GSK3β dephosphorylation induced by 6-OHDA in SH-SY5Y cells by western blot analysis. FIG. 7B shows the prevention of SchiA on GSK3β dephosphorylation induced by 6-OHDA in SH-SY5Y cells by densitometric analysis.

FIG. 8A shows the morphology of DA neurons in 6-OHDA-induced zebrafish brain with the treatment of SchiA. FIG. 8B shows quantitative analysis of TH⁺ neurons in each treatment group of FIG. 8A.

FIG. 9 shows the effect of SchiA in attenuating 6-OHDA-induced the deficit of locomotion behavior on zebrafish larval.

FIG. 10 shows the chemical structures of the five dibenzocyclooctadiene lignans of S. chinensis and benzamide antipsychotics including sulpiride and amisulpride.

FIG. 11A shows the effect of SchiA on the inhibition of MPP⁺-induced cell death in SH-SY5Y cells. FIG. 11B shows the effect of Schizandrin C (SC) on the inhibition of MPP⁺-induced cell death in SH-SY5Y cells.

FIG. 12 shows the effect of SchiA in attenuating MPTP-induced the deficit of locomotion behavior on zebrafish larval.

FIG. 13A shows the microphotographs of TH immunostaining in the SNpc of mouse brain sections. FIG. 13B shows quantification of the number of dopaminergic (TH-positive) neurons per field in the various treatment groups of FIG. 13A.

FIG. 14A shows the effect of SchiA in inhibiting MPP⁺-induced activation of caspase-3 in SH-SY5Y cells. FIG. 14B shows the effect of SchiA in inhibiting MPP⁺-induced activation of casepase-7 in SH-SY5Y cells. FIG. 14C shows the effect of SchiA on MPP⁺-induced up-regulation of caspase-3 activity.

FIG. 15A shows the effects of SchiA on Bax and Bcl-2 expression in MPP⁺-treated SH-SY5Y cells by immunoblot analysis. FIG. 15B shows the effects of SchiA on Bax and Bcl-2 expression in MPP⁺-treated SH-SY5Y cells by densitometric analysis.

FIG. 16A shows the Akt phosphorylation pathway in the neuroprotective action of SchiA on MPP⁺-induced SH-SY5Y cell damage. FIG. 16B shows the effect of Akt inhibitor IV on cytoprotection of SchiA in SH-SY5Y cells.

FIG. 17 shows the effect of SchiA on the expression levels of p-CREB, CREB and Bcl-2 and the expression levels of Bcl-2 and the p-CREB/t-CREB ratio quantified by densitometric statistical analysis.

FIG. 18A shows the Mean plasma concentration-time profile of SchiA after intravenous or intragastric administration of SchiA microemulsion at 30 mg/kg (mean±SD, n=3). FIG. 18B shows the brain and plasma concentration of SchiA detected half an hour post-intravenous administration.

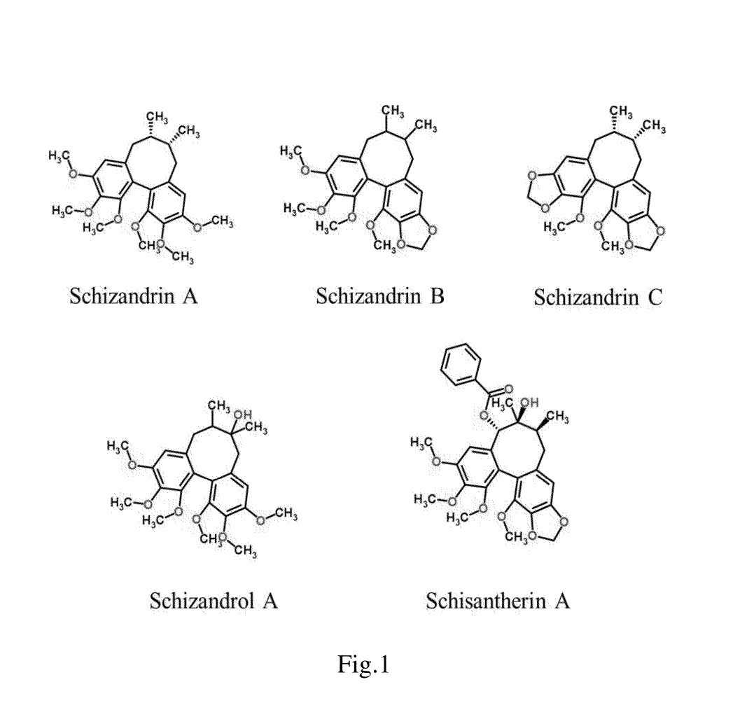

### DETAILED DESCRIPTION OF THE PREFERRED EMBODIMENTS

As used herein and in the claims, “comprising” means including the following elements but not excluding others.

L-dopa is one of the most effective medicines for PD. Although dopamine replacement may alleviate the symptoms of the disease, the medicine cannot slow down or stop the progression of neuronal degeneration. Thus, from a therapeutic point of view, an agent or a combination of agents with different mechanisms of action is needed.

Glycogen synthase kinase3 (GSK3β) belongs to the serine/threonine kinase family of proteins which is originally identified as a regulator of glycogen metabolism. GSK3β is particularly abundant in the brain. A number of kinases (Akt/protein kinase B (PKB), protein kinase A (PKA), and protein kinase C (PKC) can phosphorylate GSK3β on serine 9 (Ser9) and inactivate GSK3β (Juhaszova, Zorov et al. 2004; Juhaszova, Zorov et al. 2009). A large body of evidence suggests that GSK3β is a key activator of cell death in numerous models of neuronal apoptosis and inactivation of GSK3β by phosphorylation can promote cell viability.

The Cyclic AMP response-element binding protein (CREB) is a transcription factor involved in neuronal cell survival and differentiation (Gonzalo-Gobernado, Calatrava-Ferreras et al. 2013). CREB activation is important in transcriptional activation, leading to expression of many genes associated with cell survival (Gao, Siddoway et al. 2009). The major regulator of CREB activity is cAMP-dependent protein kinase (PKA). Increases in intracellular cAMP activated PKA act to disassociate the regulatory subunits from the catalytic subunits. Activated PKA moves into the cell nucleus, where it phosphorylates CREB (Fang, Chen et al. 2012). In its active form, CREB was shown to regulate many aspects of neuronal functioning, including neuronal excitation, development and long-term synaptic plasticity (Silva, Kogan et al. 1998). Recent evidence suggests that CREB might also be involved in an active process of neuroprotection (Walton and Dragunow 2000) or that its disruption in the brain might lead to neurodegeneration (Mantamadiotis, Lemberger et al. 2002), suggesting a pivotal role of CREB neuroprotection. However, up-regulation of CREB also plays crucial roles in regulating transcription of neuroprotective factors, including brain-derived neurotrophic factor (BDNF) and Bck-2 (Lonze and Ginty 2002). Akt is a well known signaling pathway involved in cell protection under various stresses (Wang, Sun et al. 2007). The activation of Akt regulates cell survival and prevents apoptosis and thus the activation of Akt may have pro-neuronal survival capabilities in neurodegenerative diseases (Timmons, Coakley et al. 2009). And many studies have implicated Akt activation in the neuroprotection of DA neurons in cellular and animal models of PD (Sagi, Mandel et al. 2007; Chen, Zhang et al. 2008; Lim, Kim et al. 2008; Nair and Olanow 2008). Meanwhile, Bcl-2 family plays a key role in the mitochondrial apoptotic pathway which is one of two major pathways of apoptosis. Bax and Bcl-2, the two main members of Bcl-2 family play crucial role in the cell apoptotic cascade (Cory and Adams 2002; O'Malley, Liu et al. 2003). Because the balance between the Bax and Bcl-2 can mostly impact the cell survival in the early phases of apoptotic cascade.

Schisantherin A (SchiA) and Schizandrin C (SC) are dibenzocyclooctadiene lignans isolated from the fruit of Schisandra chinensis (Turcz.) Baill (S. chinensis). In this invention, the neuroprotective effects of SchiA and SC were discovered for the first time and the underlying mechanism of the neuroprotective effect of SchiA was investigated. The present invention provides methods of treating and/or preventing neurodegenerative diseases by administrating SchiA or SC or a combination thereof. The following paragraphs will describe the methods and materials used in the experiments and the results.

### Example 1

### Materials and Methods Used in the Experiments

### Chemicals and Reagents

Schizandrin A, Schizandrin B, Schizandrin C (SC), Schizandrol A and Schisantherin A (SchiA) were purchased from National Institute for the Control of Pharmaceutical and Biological Products (NICPBP, Beijing, China). 6-hydroxydopamine (6-OHDA) was purchased from Sigma-Aldrich (Calbiochem, San Diego, Calif.). 2′,7′-dichlorofluorescein diacetate (CM-H2DCFDA) and 4-amino-5-methylamino-2′,7′-difluorofluoresecin diacetate (DAF-FM diacetate) were purchased from Molecular Probes (Eugene, Oreg., USA). MPP and MPTP were obtained from Sigma-Aldrich (Germany). MTT [3-(4,5-dimethylthiazol-2-yl)-2,5-diphenyltetrazoliumbromide] was purchased from Sigma-Aldrich (St. Louis, Mo., USA). Heat-inactivated horse serum, fetal bovine serum (FBS), penicillin and streptomycin were purchased from Gibco Invitrogen (Grand Island, N.Y., USA). Anti-TH antibody and against β-actin antibody were obtained from Milipore (USA). All other Primary antibodies and horseradish peroxidase-conjugated anti-rabbit were purchased from Cell Signaling (Danvers, Mass., USA). ECL advanced Western blotting detection kit was purchased from GE Healthcare (USA). Phosphatase inhibitor cocktail were purchased from Roche Applied Science (Indianapolis, Ind., USA). All other reagents were from Sigma-Aldrich (St. Louis USA) unless stated otherwise.

Propylene glycol and absolute alcohol were purchased from chemical reagent factory of Guangzhou. 5% polyvinyl alcohol and Tween-80 were purchased from Beyotime Institute of Biotechnology. Acetonitrile and methanol (HPLC grade) were purchased from Hanbang Company (Jiangsu, China). Ultrapure water was prepared by a Milli-Q Plus water purification system (Millipore, Bedford, Mass., USA). All other reagents were analytical grade.

### Cell Culture

Human neuroblastoma SH-SY5Y cells were purchased from the American Type Culture Collection (ATCC, Manassas, Va., USA) and cultured in a humidified 5% (v/v) CO₂ atmosphere in 37° C. in Dulbecco's Modified Eagle Medium (DMEM) (Life technologies, NY, USA) supplemented with 10% FBS, 1% penicillin (100 U/mL) and streptomycin (100 μg/mL). All experiments were carried out 48 h after the cells were seeded.

### MTT Assay

Cell viability was determined by 3-(4,5-dimethyl-2-thiazolyl) 2,5-diphenyl-2H-tetrazolium bromide (MTT) assay (Sigma-Aldrich, USA). SH-SY5Y cells were seeded in 96-well plates (1×10⁴ cells/well) for 48 h. SH-SY5Y cells were pre-treated with different concentrations of the five lignans (i.e. Schizandrin A, Schizandrin B, SC, Schizandrol A and SchiA) for 12 h and the cell viability was checked by MTT assay. Otherwise, SH-SY5Y cells were pre-treated with different concentrations of the five lignans for 12 h and then exposed to 400 μM 6-OHDA for 4 h. Cells were incubated at 37° C. for 4 h in 0.5 mg/mL MTT solution (prepared in serum free DMEM medium). The medium was then removed, and 100 μL of DMSO was added to each well to dissolve the violet-formazan crystals. The absorbance at 570 nm with 655 nm as a reference wavelength was measured by a multi-label counter (Wallac VICTOR3TMV, Perkin Elmer, Netherlands). Cell viability was expressed as a percentage of the value of the cells without 6-OHDA treatment. The LC50 and EC50 were calculated by GraphPad Prism V5.0 (GraphPad Software, Inc., San Diego, Calif.).

In another study, SH-SY5Y cells were seeded in 96-well plates (1×10⁴ cells/well) for 48 h. SH-SY5Y cells were pre-treated with different concentrations of the five lignans for 12 h and the cell viability was checked by MTT assay. SH-SY5Y cells were pre-treated with different concentrations of the five lignans for 12 h and then exposed to 2 mM MPP or vehicle for 36 h. Cells were incubated at 37° C. for 4 h in 0.5 mg/mL MTT solution (prepared in serum free DMEM medium). The medium was then discarded and 100 μL of DMSO was added to each well to dissolve the violet-formazan crystals. The absorbance at 570 nm with 655 nm as a reference wavelength was measured by a multi-label counter (Wallac VICTOR3TMV, Perkin Elmer, Netherlands). Cell viability was expressed as a percentage of the value of the cells without MPP treatment. The LC50 and EC50 were calculated by GraphPad Prism V5.0 (GraphPad Software, Inc., San Diego, Calif.).

### ROS Production

SH-SY5Y cells seeded at 96-well plates (1×10⁴ cells/well) were incubated with 5 μM fluorescent probe CM-H2DCFDA at 37° C. for 20 min after treatment with SchiA or 6-OHDA. The fluorescence intensity was determined by multi-label counter at wavelengths of excitation at 493 nm and emission at 522 nm.

### NO Staining

Intracellular NO was evaluated by using the fluorescent probe 4-amino-5-methylamino-2′,7′-difluorofluoresecin diacetate (DAF-FM diacetate), which is cell-permeant and diffuses passively across cellular membranes. Once inside the cells, DAF-FM diacetate is deacetylated by intracellular esterases to DAF-FM. DAF-FM is essentially non-fluorescent until DAF-FM reacts with NO. Thus, it can be used to quantify intracellular NO production. The cells were seeded in 96-well, black bottom-clear plates. After treatment with 60 μM SchiA for 12 h and then exposure to 6-OHDA for 1 h, cells were washed in PBS and incubated for 20 min at 37° C. in darkness in a medium containing 1% serum plus 2.5 μM DAF-FM diacetate (diluted in PBS). The cells were then washed twice in PBS and the fluorescence was evaluated in a multi-label counter at an excitation wavelength of 495 nm and an emission wavelength of 515 nm. The increase in fluorescence for each treatment was calculated as the relative fluorescence of each treatment compared with the untreated control cells.

### Western Blot Analysis

After treatment, SH-SY5Y cells were washed three times with PBS and then lysis with RIPA lysis buffer containing 1% PMSF and 1% Protease Inhibitor Cocktail and incubated for 30 min on ice. Cell lysates were centrifuged at 12,500 g for 20 min at 4° C. The supernatant was separated and the amount of protein was determined using the BCA protein assay kit (Thermo Scientific Pierce, USA). Protein samples (30 μg) were separated by SDS-PAGE (10% (w/v)) polyacrylamide gel and then transferred to a polyvinylidene difluoride (PVDF) membrane (Bio-Rad, Hercules, Calif.). Subsequently, the membrane was blocked with 5% (v/v) non-fat milk in PBST (PBS containing 0.1% Tween 20) for 1 h at room temperature. The blots were incubated overnight at 4° C. with primary antibodies. After three washes with PBST, the membranes were incubated with horseradish peroxidase-conjugated secondary antibodies (1:2000) in PBST with 5% non-fat milk for 1 h at room temperature. After repeated washes, proteins were visualized with an ECL advanced western blotting detection kit. Photographs of protein bands were taken by a Molecular Imager ChemiDoc XRS (Bio-Rad, Hercules, Calif.). Quantitative assessment of protein bands was done with Gel Doc™ XRS equipped with Quantity One software.

### Caspase-3 Activity Assay

Caspase-3 activity was detected using the EnzChek® Caspase-3 Assay Kit according to the manufacturer's protocol. In brief, the reaction mixture (total volume 100 μL) contained 50 μL of cell lysate and 50 μL of caspase-3 substrate (Z-DEVD-R110 substrate; the final concentration is 5 mM) in assay buffer, and the assay was carried out in a 96-well plate. To account for non-specific hydrolysis of substrate, the samples were incubated at room temperature for approximately 30 minutes and measured the fluorescence (excitation/emission˜496/520 nm).

### Fish Maintenance and Drug Treatment

The wild type zebrafish was used for this study. Embryos were collected after natural spawning, staged according to standard criteria and raised synchronously at 28.5° C. in embryo medium (13.7 mM NaCl, 540 μM KCl, pH 7.4, 25 μM Na2HPO4, 44 μM KH2PO4, 300 μM CaCl2, 100 μM MgSO4, 420 μM NaHCO3, pH 7.4). Zebrafish were staged by days post fertilization (dpf) according to criteria. Ethical approval for the animal experiments was granted by the Animal Research Ethics Committee, University of Macau. Drugs (i.e. the five lignans) were dissolved in DMSO and directly added into the zebrafish embryo medium to treat fish. The final concentration of DMSO was always less than 0.5%, which showed no toxicity to zebrafish. An equal concentration of DMSO in embryo medium was used as vehicle control in each experiment.

### Anti-Tyrosine Hydroxylase (TH) Whole-Mount Immunostaining

Zebrafish at one dpf were exposed to 250 μM 6-OHDA in the presence or absence of 10 μM Schi A or 3 μM nomifensine (Nom, a DAT (dopamine transporter) inhibitor used as a positive control) for 2 days. Then zebrafish were collected for immunostaining to detect the DA neurons. Zebrafish were fixed in 4% paraformaldehyde in PBS for 30 min, rinsed, and stored at −20° C. in 100% MtOH. Whole-mount immunostaining was done by standard methods (Zhang, Cheang et al. 2011). Briefly, paraformaldehyde fixed samples were blocked (2% goat serum and 0.1% BSA in PBST,) for 1 h at room temperature. A mouse monoclonal anti-tyrosine hydroxylase antibody (1:200 diluted in blocking buffer) was used as the primary antibody and incubated overnight with the sample at 4° C. The next day, samples were washed 6 times with PBST (30 min each wash), followed by incubation with a fluorescent secondary antibody as per manufacturer's guidelines. After staining, zebrafish were flat-mounted with 3.5% methylcellulose and photographed. Semi-quantification TH⁺ cells were assessed by an investigator blinded to drug treatment history of zebrafish using Image-Pro Plus 6.0 software (Media Cybernetics, Silver Spring, Md., USA). Results are expressed as percentage of area of TH⁺ cells in untreated control group.

### Locomotion Behavioral Test of Zebrafish

Zebrafish behaviour was analyzed using a digital video tracking system (Viewpoint ZebraLab system). The system consists of a digital video camera connected to a computer system running the analysis software ZebraLab Man Rev 3.6B. The 3 dpf zebrafish larvae were under co-treatment of 150 μM 6-OHDA and various concentrations of SchiA for 4 days. Then the total distance of movement was recorded using a 96-well plate filled with 200 μl embryo medium in a 10 minutes long session. Zebrafish were allowed to accommodate to the environment of the system for 1 hour before the start of the data acquisition.

In another study, the 3 dpf zebrafish larvae were under co-treatment of 10 μM MPTP and various concentrations of SchiA for another 4 days. Then the total distance of movement was defined as the distance (in cm) was recorded using a 96-well plate filled with 200 μL embryo medium in a 10 minutes long session. Zebrafish were allowed to accommodate to the environment of the system for 1 hour before the start of the data acquisition.

### MPTP-Induced Parkinson's Disease Model in Mice

Male C57BL/6 mice (20±2 g body weight, 6-8 weeks of age) were purchased from the Laboratory Animal Center of Guangdong Province and were fed six animals per cage, under a 12 h light/dark cycle with ad libitum access to food and water. Mice were handled daily and allowed 3 days to acclimate before each treatment. Six groups of mice (n=7/group, total 42 mice) were assigned for the neuroprotective study in vivo. SchiA at different concentrations (30, 100 or 300 mg/kg/day), positive control drug (Selegiline, 10 mg/kg/day) or vehicle (Tween-80/ml propylene glycol/alcohol/5% PVA, 1:1:1:17, v/v/v/v) were administered by intragastric gavage (i.g.) daily for 14 days. Then mice were injected by intraperitoneal (i.p.) with 30 mg/kg MPTP hydrochloride (Sigma-Aldrich) or physiological saline daily on days 15-19 (total 5 days). In order to allow for the full conversion of MPTP to its active metabolite MPP⁺, a further 3 days of resting period followed (Levites, Weinreb et al. 2001). All animal experiments were conducted according to the ethical guidelines for animal experiment of Jinan University. The experimental protocols were approved by the Ethics Committee for Animal Experiments of Jinan University (permit number 20110810).

### Tissue Processing and Tyrosine Hydroxylase (TH) Immunohistochemistry

Three days after MPTP treatment, animals were anesthetized by i.p. administration of 40 mg/kg chloral hydrate (10% (w/v) dissolved in distilled water) and perfused intracardially with 100 mL PBS (0.1 mmol/L, pH 7.4) followed by 150 mL 4% paraformaldehyde (PFA) in PBS. After intracardial perfusion, brains were collected and post-fixed in 4% PFA for another 24 h at 4° C., embedded in paraffin, and cut into 6 μm coronal sections encompassing the entire SNpc. As the decrease of TH-positive neurons in response to MPTP treatment is the most prominent at medial levels of the SNpc (Hayley, Crocker et al. 2004), the inventors selected the sections from the area encompassed between −3.08 and −3.20 from bregma to perform immunohistochemisty. Immunohistochemistry of brain tissues was performed as previously described (He, Yamauchi et al. 2008; Yokoyama, Takagi et al. 2008) with minor modifications. Sections were deparaffinized in xylene and rehydrated in a graded ethanol series. Sections were incubated with 3% hydrogen peroxide (H₂O₂) for 10 minutes at room temperature to inactive endogenous peroxidase activity followed by antigen retrieval in citrate buffer for 15 minutes in a microwave oven at 95° C. Non-specific protein binding was blocked with 10% bovine serum in PBS (0.01 M, pH 7.4). Between each treatment, the slides were washed at least three times with deionized water for 5 minutes. Sections were then incubated for 1 h at room temperature with a rabbit anti-mouse TH polyclonal antibody (1:1000; Millipore, USA) diluted in Immunol Staining Primary Antibody Dilution Buffer. Then the sections were incubated with a biotinylated HRP-conjugated secondary antibody for 30 minutes at room temperature. TH-positive neurons were then visualized using a DAB Kit according to the manufacturer's instructions (Shanghai, Gene Company, China). The peroxidase reaction was stopped after 30 s. Sections were counterstained with Improved-Hematoxylin for 1 minute. Finally, sections were cover-slipped with neutral balsam. The results were analyzed by counting the numbers of TH-positive cells at ×10 magnifications on a stereomicroscope (BX51, Olympus Corp. Japan). TH-positive cells in 6 position-matched sections of each mouse were counted manually by operator who was blinded to the drug treatment. The average number of TH-positive cells per section was used to represent dopaminergic neuron livability.

### Assay Application for SchiA Pharmacokinetics Study

Male Sprague-Dawley rats (SD rats) with body weight between 220 and 250 g and age between 6 and 7 weeks were supplied by Guangdong Medical Experiment Animal Center (Guangzhou, China). SchiA was prepared as microemulsion for i.v. and p.o. at 30 mg/kg. The concentration of SchiA microemulsion was 4.9 mg/ml. After administration of SchiA, blood samples were collected at 0.033, 0.167, 0.5, 1, 2, 4, 8, 12, 24 and 36 h, respectively. Plasma samples were obtained after centrifugation of the collected blood samples at 10,000 rpm for 5 min. Subsequently, in 100 μl plasma which was taken from the supernatant, 200 μl of 50% acetonitrile and 50% methanol were added. After vortexing for 20 s and centrifuging at 12,000 rpm for 10 min, the supernatant was collected and filtered through a 0.22 μm membrane, of which 20 μl was injected into the chromatographic system for analysis.

### Blood Brain Barrier Experiment

To investigate the capability of permeation of blood brain barrier (BBB). On the experimental day, heart perfusion was chosen to clear blood interference at brain under 10% chloralic hydras (i.p. 250 mg/kg) anesthesia, and brain was collected at scheduled time after dosing.

Brain tissues were thawed and homogenized in precooled saline (at the 1:2 g:ml ratio). 250 μl of homogenized solution was added 200 μl acetonitrile and 150 μl methanol. After vortexing for 20 s, centrifugations at 12,000 rpm for 10 min were for the separation. The denatured protein was separated and the supernatant was collected and filtered through a 0.22 μm membrane. A 20 μl volume of the supernatant was injected into HPLC for analysis.

Quantitative analysis of SchiA was completed on an Agilent series 1260 HPLC apparatus (Agilent Technologies, Santa Clara, Calif., USA) equipped with a vacuum degasser, a quaternary pump, a manual sampler and an ultraviolet detector. RP Phenonenex C-18 column (250 mm×4.6 mm, 5 μm) was used to separate at 35° C. with a mobile phase of water and methanol (83:17, v/v). The flow rate was 1 ml/min. The monitoring wavelength of SchiA was 230 nm.

### Statistical Analysis

Measurements were done 3 times independently for multiple biological samples. The data were analyzed using GraphPad Prism V5.0 (GraphPad Software, Inc., San Diego, Calif.). One-way analysis of variance (ANOVA) and Dunnett's test were used to evaluate the statistical differences. The value of statistical significance was set at p<0.05.

### Example 2 Study on Neuroprotective Effect of SchiA on SH-SY5Y Cells Against 6-OHDA-Induced Cytotoxicity

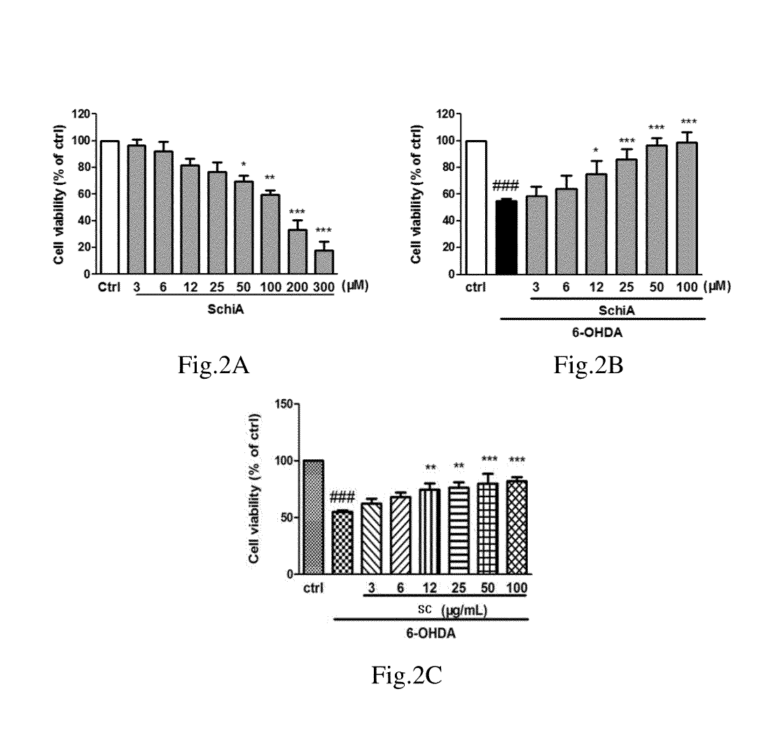

To evaluate the cytotoxicity of five selected lignans of S. chinensis, SH-SY5Y cells were treated with various concentrations of the tested compounds for 12 h and the cell viability was measured using the MTT assay. In FIG. 2A, treatment with 3-25 μM SchiA did not have any detectable toxicity on the SH-SY5Y cells. The LC₅₀ values of the five selected ligans including Schizandrin A, Schizandrin B, SC, Schizandrol A and SchiA were 232.6, 189.3, 134.8, 154.6, and 120.7 μM, respectively.

To further study the neuroprotective activities of the five lignans against 6-OHDA-induced cytotoxicity, cells were treated with various concentrations (3, 6, 12, 25, 50 and 100 μM) of the tested compounds for 12 h before exposed to 400 μM 6-OHDA for 4 h. As shown in FIG. 2B, the cell viability of 6-OHDA group without treatment of SchiA was reduced significantly to 50% compared to the control group. Pretreatment with SchiA protected SH-SY5Y cells against 6-OHDA-induced damage in a dose-dependent manner. As shown in FIG. 2C, the cell viability of 6-OHDA group without treatment of SC was reduced significantly to 50% compared to the control group. Pretreatment with SC protected SH-SY5Y cells against 6-OHDA-induced damage in a dose-dependent manner. However, the rest of lignans including Schizandrin A, Schizandrin B and Schizandrol A did not show protective effects. The EC₅₀ values and therapeutic index of SchiA were 8.1 μM and 14.9 (see table 1) while the EC50 values and therapeutic index of SC were 15.3 μM and 8.8 (see table 1). SchiA exhibited the strongest neuroprotective effect with the lower cytotoxicity among tested lignans. The results further show that SchiA and SC can be used to treat and/or prevent neurodegenerative disease, preferably the disease caused by dopaminergic neuron loss.

TABLE 1
LC₅₀, EC₅₀ and Therapeutic Index of five dibenzocyclooctadiene lignans
of S. chinensis.

| Name | LC₅₀ (μM) | EC₅₀ (μM) | Therapeutic Index |
|---|---|---|---|
| Schizandrin A | 232.6 | | |
| Schizandrin B | 189.3 | | |
| SC | 134.8 | 15.3 | 8.8 |
| Schizandrol A | 154.6 | | |
| SchiA | 120.7 | 8.1 | 14.9 |

### Example 3

### Study on the Effect of SchiA in Attenuating 6-OHDA-Induced Accumulation of ROS

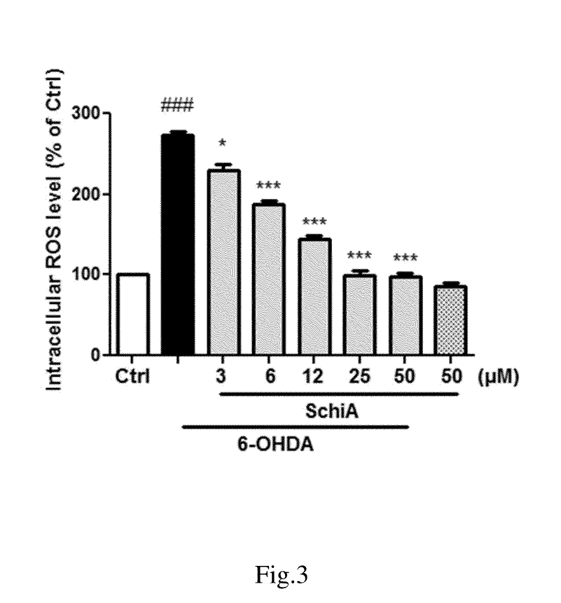

The generation of excess ROS by auto-oxidation of 6-OHDA, considered to be involved in 6-OHDA-induced cellular injury (Cohen and Heikkila 1974). Therefore, to examine whether SchiA prevented the production of ROS from 6-OHDA, the accumulation of ROS was measured by fluorescent probe CM-H2DCFDA in SH-SY5Y cells. The fluorescence intensity of CM-H2DCFDA was measured after SH-SY5Y cell were pretreated with different concentrations of SchiA for 12 h and then treated with or without 400 μM 6-OHDA for 4 h. The results of FIG. 3 are expressed as a percentage of that of the control group. ###P<0.005 versus control group; *p<0.05, **p<0.01 and *** p<0.005 versus 6-OHDA group. As shown in FIG. 3, 6-OHDA induced a 3-fold increase in the intracellular ROS level in SH-SY5Y cells. The increased level of ROS induced by 6-OHDA was significantly reduced by SchiA in a dose-dependent manner (3, 6, 12, 25 and 50 μM). Additionally, SchiA alone had no obvious effect on intracellular ROS levels. These data suggest that SchiA possesses the cytoprotective effect against 6-OHDA-induced cell death and accumulation of ROS in SH-SY5Y cells.

### Example 4

### Study on the Effect of SchiA in Attenuating 6-OHDA-Induced NO Over-Production and iNOS Over-Expression in SH-SY5Y Cells

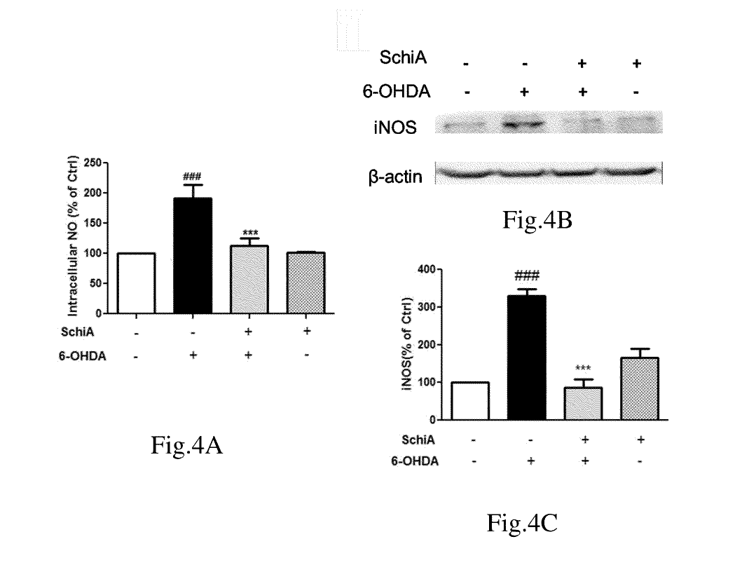

Augmented NO production subsequent to iNOS induction appears to play an important role in the initial phase of 6-OHDA-induced neuro-damage models in vitro and in vivo (Singh, Das et al. 2005; Lin, Uang et al. 2007; Shih, Chen et al. 2011). In this study, SH-SY5Y cells were pretreated with 60 μM SchiA for 12 h before incubating with 400 μM 6-OHDA for 2 h. Expression of the total iNOS proteins were determined by Western blot analysis. Intracellular NO was stained by the fluoresecnt indicator DAF-FM diacetate. As shown in FIG. 4A, exposure of SH-SY5Y cells to 400 μM 6-OHDA for 2 h led to a rapid increase in DAF-FM fluorescence, almost 2-fold compared with the control group (P<0.005). SchiA (60 μM) pre-treatment inhibited such increase in DAF-FM fluorescence, while SchiA alone had no effect on DAF-FM fluorescence intensity (FIG. 4A). Results of FIGS. 4A-C are expressed as a percentage of relative fluorescent intensity (RFI) of the Control group. ###P<0.005 versus control group; *p<0.05, **p<0.01 and *** p<0.005 versus 6-OHDA group. These results show that SchiA has the neuroprotective effects on 6-OHDA-induced neuro damage. These results may also indicate that the protective effect of SchiA on 6-OHDA-induced SH-SY5Y cell viability is through prevention of elevation in intracellular NO levels.

Furthermore, Western blot results reflected that 400 μM 6-OHDA for 2 h induced a 2-fold increase in the immunoreactivity of iNOS in SH-SY5Y cells compared with control group. Pre-treatment with 60 μM SchiA reduced the expression of iNOS induced by 6-OHDA significantly. (FIGS. 4 B and 4C). Additionally, Treatment with 60 μM SchiA alone had no obvious influence on the expression of iNOS in SH-SY5Y cells. Pretreatment with SchiA also results in a down-regulation of 6-OHDA-induced iNOS over-expression, similar to the inhibitory effect observed in 6-OHDA-induced NO overproduction. These results show that SchiA has the neuroprotective effects on 6-OHDA-induced neuro damage. These results also imply that SchiA attenuated NO overproduction via down-regulation of iNOS over-expression in 6-OHDA-treated SH-SY5Y cells.

### Example 5

### Study on the Effect of SchiA on 6-OHDA-Induced p-ERK Regulation

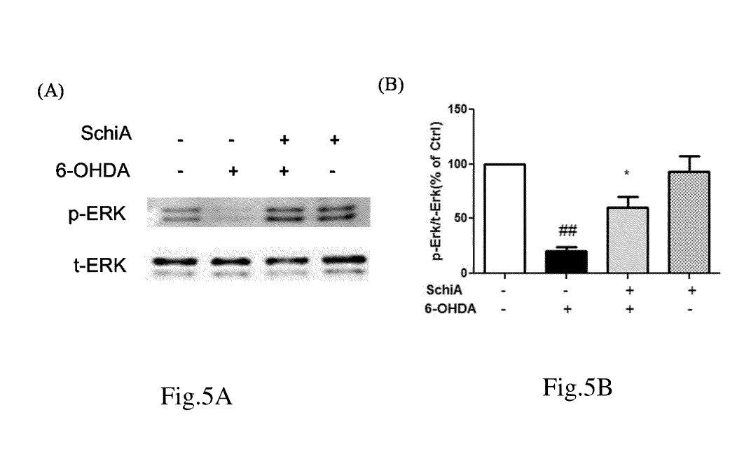

The inventors further performed experiments to address the involvement of MAPK pathways, which are associated with oxidative stress induced cell death and cell survival in the neuroprotective action of SchiA. Cells were pretreated with 60 μM SchiA for 12 h before incubating with 400 μM 6-OHDA for another 4 h. Cells were lysed and examined by western blot. 6-OHDA induced a significant decrease in the immunoreactivity of phosphorylated ERK at 4 h in SH-SY5Y cells (FIGS. 5A and 5B). Pretreatment with SchiA prevented the reduction in ERK1/2 phosphorylation induced by 6-OHDA dramatically (FIGS. 5A and 5B). ##P<0.01 versus control group; *p<0.05, **p<0.01 and *** p<0.005 versus 6-OHDA group. Treatment with 60 μM SchiA alone did not change the levels of phosphorylated ERK1/2. Total levels of ERK1/2 (nonphosphorylated and phosphorylated) were not affected by any of the treatments.

### Example 6

### Study on Modulation of the Akt Signaling Pathway Involved in Neuroprotective Effect of SchiA

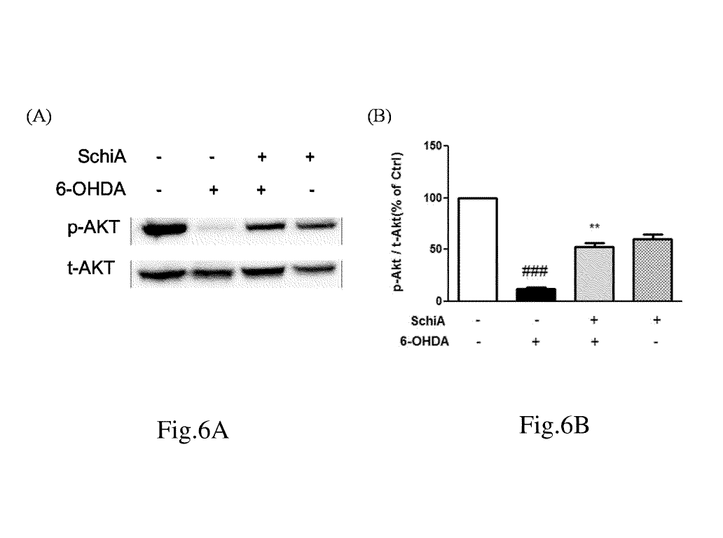

The inventors also investigated the contribution of the Akt pathway in the neuroprotective effects of SchiA. The inventors measured phosphorylation-Akt (p-Akt) levels after pre-treated with 60 μM SchiA for 12 h and then exposed to 400 μM 6-OHDA for another 4 h (FIGS. 6A and 6B). The immunoblot showed that pretreatment with SchiA prevented the reduction in p-Akt level induced by 6-OHDA dramatically (FIGS. 6A and 6B). ##P<0.01 versus control group; *p<0.05, **p<0.01 and *** p<0.005 versus 6-OHDA group. Treatment with 60 μM SchiA alone did not change the levels of p-Akt level. Total levels of Akt (nonphosphorylated and phosphorylated) were not affected by any of the treatments. These results indicated that SchiA could activate Akt signaling pathway to regulate the cytoprotective effect.

### Example 7

### Study on the Mediation of GSK3β in the Neuroprotective Effect of SchiA Against 6-OHDA-Induced Cell Death

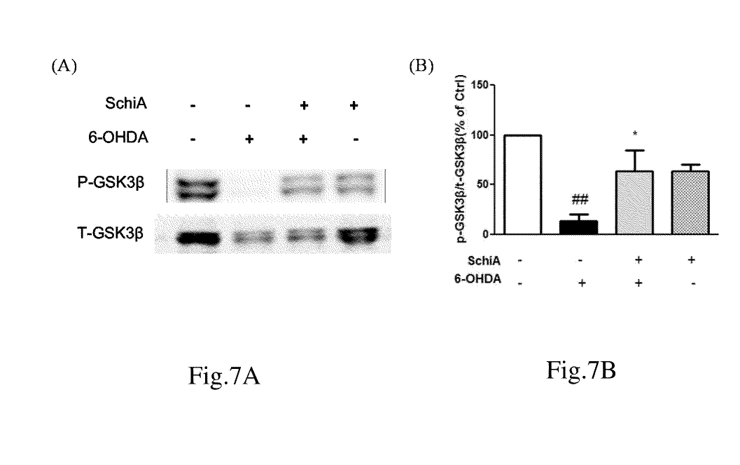

GSK3β is a kinase that plays a pivotal role in numerous cellular functions ranging from glycogen metabolism and modulation of microtubule dynamics to the regulation of cell survival (Grimes and Jope 2001; Kaytor and Orr 2002). In this study, cells were pretreated with SchiA for 12 h before incubating with 400 μM 6-OHDA for another 4 h. Cells were lysed and examined by western blot. As shown in FIGS. 7A and 7B, the results depicted that 6-OHDA incubation for 4 h decreases GSK3β phosphorylation at serine 9 residue. However, pretreatment with SchiA completely blocked the repression of GSK-3β phosphorylation effect of induced by 6-OHDA. ##P<0.01 versus control group; *p<0.05, **p<0.01 and *** p<0.005 versus 6-OHDA group. This result suggests the neuroprotective effect of SchiA against 6-OHDA-induced cell death is also, partially mediated by GSK3β.

### Example 8

### Study on the Prevention of SchiA Against the Dopaminergic Neuron Loss Induced by 6-OHDA in Zebrafish

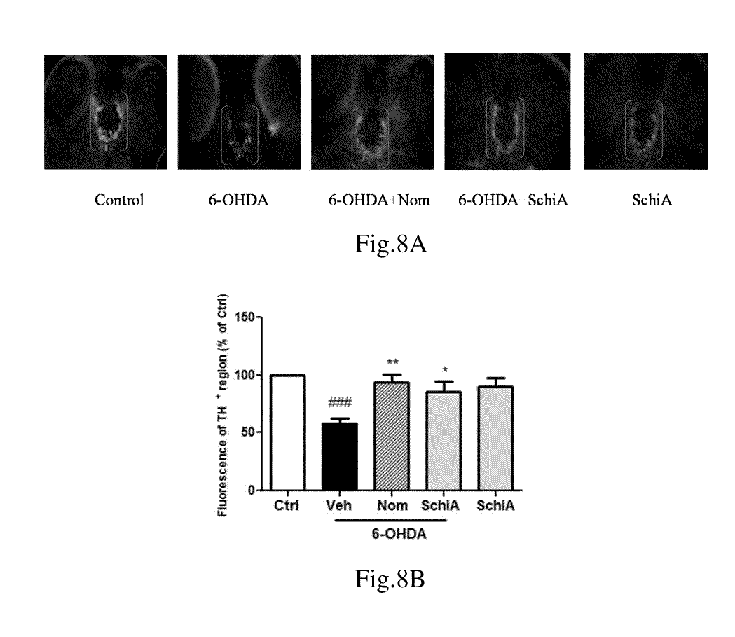

To further evaluate the neuroprotective effect of SchiA in vivo, the inventors examined the DA neurons in zebrafish by whole-mount immunofluorescent staining with an antibody against tyrosine hydroxylase (TH). As shown in FIGS. 8A and 8B, after 6-OHDA treatment, the area of TH-immunoreactive regions observed in the diencephalons of zebrafish (indicated by brackets) were decreased dramatically. Data is expressed as percentage of the control group. Values represent the mean±SEM of 15 fish larvae per group of 3-time independent experiments; ###p<0.005 versus Control group; *p<0.05, **p<0.01 and *** p<0.005 versus 6-OHDA group. Importantly, co-treatment with 10 μM SchiA could attenuate the dopaminergic neuron loss induced by 6-OHDA almost to the normal level, suggesting that SchiA provides the protective effect on 6-OHDA-induced dopaminergic neuron death in zebrafish and further showing that SchiA can prevent and/or treat neurodegenerative disease caused by dopaminergic neuron loss, such as Parkinson's disease. Nomifensine, a DAT inhibitor, was used as the positive control and was shown to exert considerable protection against 6-OHDA-induced DA neuron loss.

### Example 9

### Study on the Inhibitory Effect of SchiA on 6-OHDA-Induced Decrease of Total Distance of Movement in Zebrafish

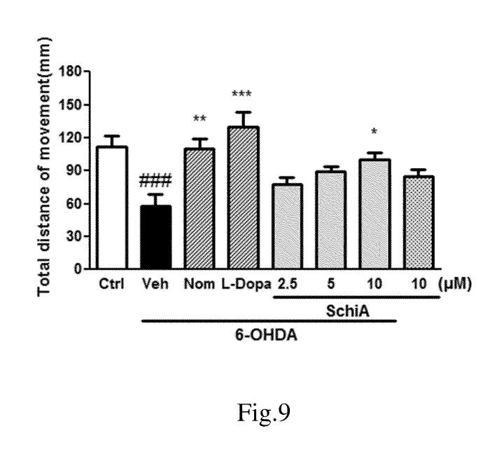

Moreover, 6-OHDA treatment also markedly altered the swimming behavior of zebrafish. In this study, zebrafish larvae at 3 dpf were exposed to 2.5-10 μM SchiA with or without 250 μM 6-OHDA for another 4 days, and zebrafish larval co-treated with 6-OHDA and 150 μM L-dopa or 3 μM NOM (Nomifensine) were used as positive controls. After treatment, zebrafish were collected to perform locomotion behavior test using Viewpoint Zebrabox system and total distances travelled in 10 min were calculated. Values in FIG. 9 represent the mean±SEM of 12 fish larvae per group of 3-time independent experiments. ###P<0.005 versus control group; *p<0.05, **p<0.01 and *** p<0.005 versus 6-OHDA group. Zebrafish embryos exposed to 6-OHDA between 3 dpf and 7 dpf displayed a huge impact on swimming behavior. Typical swimming paths of untreated control and 6-OHDA-treated zebrafish larvae are shown in FIG. 9. The total swimming distance in 10 min travelled by the zebrafish larvae was decreased after exposure to 6-OHDA. SchiA inhibited 6-OHDA-induced reduction of total distance of movement by zebrafish in a dose dependent manner and 10 μM SchiA treated alone did not significantly affect the locomotor behavior of normal zebrafish. And SchiA was as effective as nomifensine and L-dopa as the positive control drugs, which rescued the deficit of locomotor activity of 6-OHDA-treated zebrafish to normal. The results show that SchiA can be used to treat/prevent neurodegenerative disease, preferably the neurodegenerative caused by dopaminergic neuron loss, and more preferably Parkinson's disease.

### Example 10

### Study on the Protection Effect of SchiA and SC on SH-SY5Y Cells Against MPP⁺-Induced Cell Damage

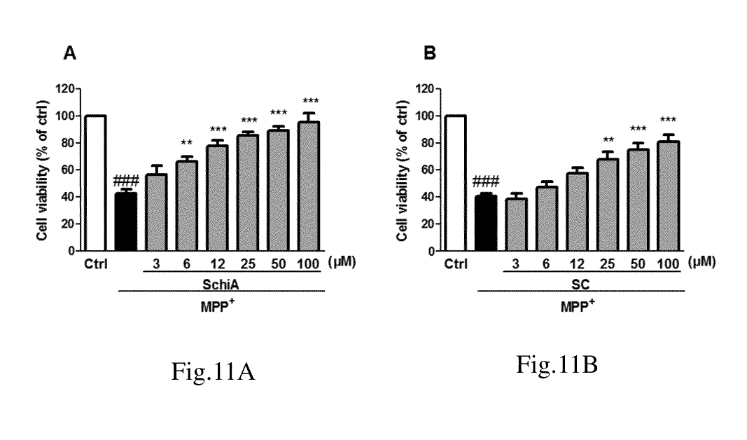

To evaluate the cytotoxicity of five selected lignans of S. chinensis, SH-SY5Y cells were treated with various concentrations of the tested compounds for 12 h and the cell viability was measured using the MTT assay. Treatment with 3-25 μM SchiA and SC did not have any detectable toxicity on the SH-SY5Y cells. In table 1, the LC₅₀ values of the five selected lignans including Schizandrin A, Schizandrin B, SC, Schizandrol A and SchiA were 232.6, 189.3, 134.8, 154.6, and 120.7 μM, respectively. To further study the neuroprotective activities of the five lignans in vitro, cells were treated with various concentrations of the tested compounds for 12 h before exposed to MPP⁺. As shown in FIG. 11A, the cell viability of MPP group without SchiA treatment was reduced significantly to 40.7% compared to the control group and changed the cell morphology. SchiA and SC can both protect against MPP⁺-induced cell damage in a dose-dependent manner (FIGS. 11A and 11B). Values represent mean±S.D. ###P<0.005 versus control group; *p<0.05, **p<0.01 and *** p<0.005 versus MPP group. However, the rest of lignans including Schizandrin A, Schizandrin B and Schizandrol A did not show protective effects. The EC₅₀ values of SchiA and SC were 8.1 and 15.3 μM, respectively. The therapeutic index of SchiA and SC were 14.9 and 8.8, respectively. The results show that SchiA and SC can be both used to treat and/or prevent neurodegenerative diseases, such as Parkinson's disease.

### Example 11

### Study on the Inhibition Effect of SchiA on MPTP-Induced Decrease of Total Distance of Movement in Zebrafish

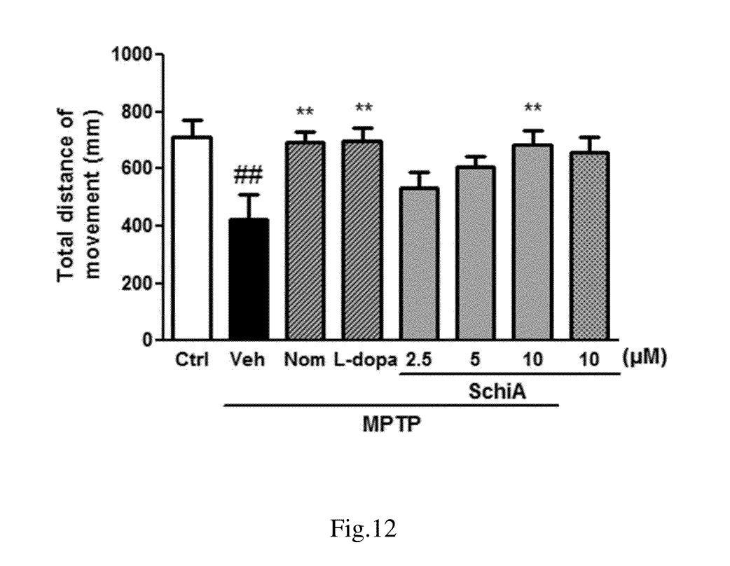

In this study, Zebrafish larvae at 3 dpf were exposed to 2.5-10 μM SchiA with or without 10 μM MPTP for another 4 days, and zebrafish larval co-treated with MPTP and 150 μM L-dopa or 3 μM NOM (Nomifensine) were used as positive controls. After treatment, zebrafish were collected to perform locomotion behavior test using Viewpoint Zebrabox system and total distances travelled in 10 min were calculated. Data were the mean 6 SEM of 12 fish larvae per group from 3-time independent experiments. ##P<0.01 versus control group; * P<0.05 and ** P<0.01 versus MPTP group. As shown in FIG. 12, MPTP markedly altered the swimming behavior of the zebrafish as a consequence of DA neuronal injury and the total distance travelled by the zebrafish larvae decreased significantly. SchiA inhibited MPTP-induced reduction of total distance of movement in zebrafish in a dose dependent manner and 10 μM SchiA treated alone did not significantly affect the locomotor behavior of normal zebrafish. At the same condition, SchiA was as effective as nomifensine and L-dopa which were the positive control drug. SchiA inhibited the deficit of locomotor activity of MPTP-treated zebrafish to normal. Thus the result shows that SchiA can be used to treat and/or prevent neurodegenerative disease, preferably the disease caused by dopaminergic neuron loss and more preferably Parkinson's disease.

### Example 12

### Study on the Prevention of SchiA Against TH Neurons of SNpc Loss Induced by MPTP on Mice Brain in a Dose-Dependent Manner

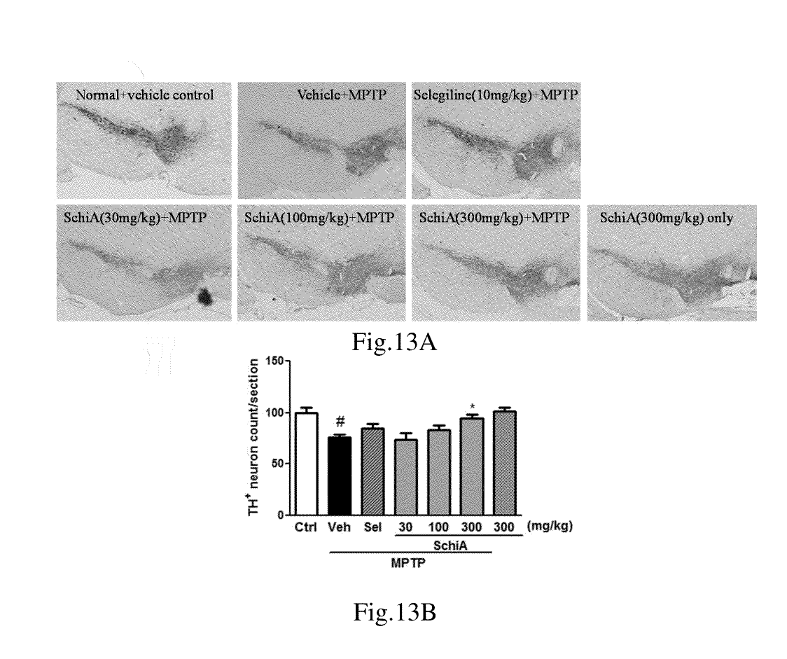

The inventors further investigated the in vivo neuroprotective effects of SchiA on a MPTP-injected mice model of PD. Zebrafish larvae at 3 dpf were exposed to 2.5-10 μM SchiA with or without 10 μM MPTP for another 4 days, and zebrafish larval co-treated with MPTP and 150 μM L-dopa or 3 μM NOM (Nomifensine) were used as positive controls. After treatment, zebrafish were collected to perform locomotion behavior test using Viewpoint Zebrabox system and total distances travelled in 10 min were calculated. Data were the mean 6 SEM of 12 fish larvae per group from 3-time independent experiments. In this study, co-treated 10 mg/kg Selegiline with MPTP was used as positive control group. Data were expressed as mean±S.D. (n=6). #P<0.05 versus control group; *P<0.05 and **P<0.01 versus MPTP group. As shown in the FIGS. 13A and 13B, MPTP intoxication remarkably reduced the number of dopaminergic neurons visualized by immunostaining of TH expression in the SNpc, which decreased from average 92±14 cells/section in normal control to 71±14 cells/section in vehicle plus MPTP treatment (p<0.05 compared with normal control). SchiA at 300 mg/kg treatment significantly protected against MPTP-induced loss of TH-positive neurons, which remained 82±12 cells/section (p<0.05 compared with vehicle plus MPTP treatment group). 300 mg/kg of SchiA was approximately as effective as the MAO-B inhibitor and clinical anti-PD drug selegiline, which at 10 mg/kg retained TH-positive neurons at 80±15 cells/section (p<0.05 compared with vehicle plus MPTP treatment group). However, SchiA at 30 and 100 mg/kg did not exert statistical significant neuroprotective effect. SchiA at 300 mg/kg treatment alone did not obviously alter the number of TH-positive neurons.

### Example 13

### Study on the Effect of SchiA in Attenuating MPP⁺-Induced Caspase 3 and Caspase 7 Activation in SH-SY5Y Cells

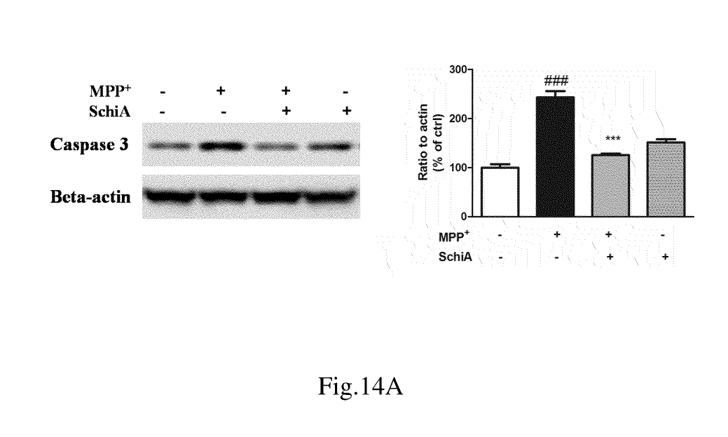

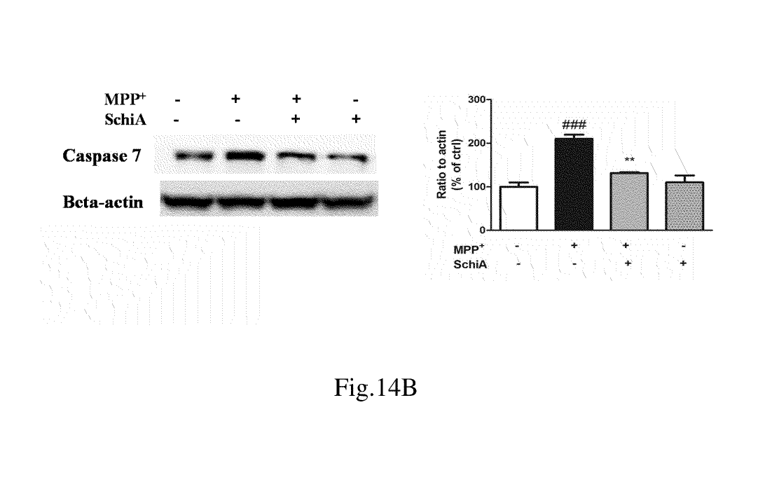

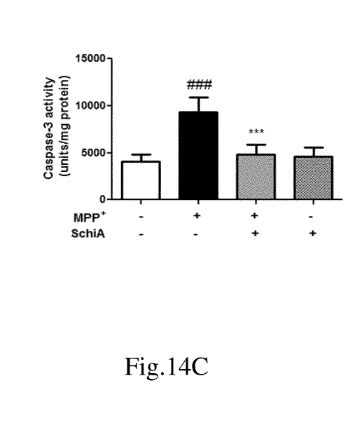

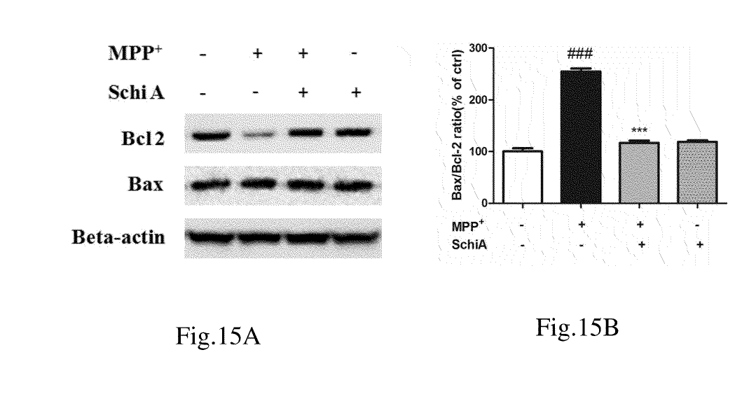

The inventors performed Western blot to measure the expression of active-form of caspase-3 and caspase-7 in SH-SY5Y cells. Cells were pretreated with 60 μM SchiA for 12 h and then treated with 2 mM MPP for another 36 h. Cells were lysed and examined by western blot. As shown in FIGS. 14A and 14B, the expression for active form of caspase-3 and caspase-7 was significantly up-regulated by MPP treatment. In contrast, SH-SY5Y cells that were pre-treated with SchiA exhibited a significant decrease in MPP⁺-induced caspase-3 and caspase-7 activation. Colorimetric assay of caspase-3 activity also indicated that SchiA dramatically suppressed the elevation of caspase-3 activity caused by MPP (FIG. 14C). Caspase-3 activity was expressed as units per mg protein. ###p<0.005 versus control group; *p<0.05, **p<0.01 and *** p<0.005 versus MPP+ group.

### Example 14 Study on the Inhibitory Effect of SchiA on MPP⁺ Induced Increases in the Bax/Bcl-2 Ratio

To investigate whether SchiA has any effect on the expression of Bax and Bcl-2 proteins in MPP⁺-induced SH-SY5Y cells, the expression levels of Bax and Bcl-2 were determined by Western blot analysis. SH-SY5Y cells were pretreated with 60 μM SchiA for 12 h before incubating with 2 mM MPP for 36 h. The expression of Bax or Bcl-2 in cell lysates was determined by Western blot analysis. As shown in FIGS. 15A and 15B, Bax protein expressions did not changed significantly in 2 mM MPP or 60 μM SchiA treatment groups compared with control group. However, 2 mM MPP treatment could decrease the Bcl-2 expression level significantly and SchiA could inhibit the down-regulation of Bcl-2 expression induced by MPP⁺. Interestingly, SchiA treated alone could also increase the expression of Bcl-2. The Bax/Bcl-2 ratio increased to 2.5-fold of control upon treatment with MPP⁺, while SchiA effectively prevented the MPP⁺-induced increase of the Bax/Bcl-2 ratio. In this study, β-actin was shown as an internal control. In FIGS. 15A-B, the Bax/Bcl-2 ratio are expressed as a percentage of that of the control group. ###P<0.005 versus control group; ***P<0.005 versus MPP group.

### Example 15

### Study on the Modulation of the PI3K/Akt Signaling Pathway Involved in the Neuroprotective Effect of SchiA

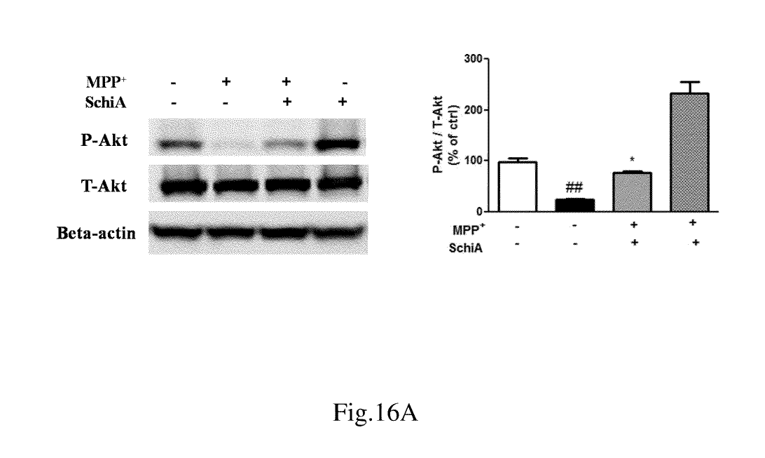

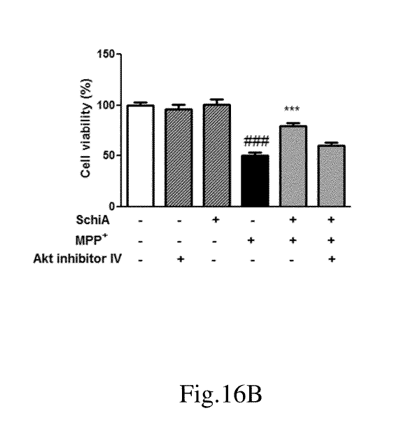

The inventors also investigated the contribution of the PI3K/Akt pathway in the neuroprotective effects of SchiA. The inventors measured phosphorylation-Akt (p-Akt) levels after pre-treated with 60 μM SchiA for 12 h and then exposed to 2 mM MPP for another 36 h. As shown in FIG. 16A, SchiA treated alone could up-regulate p-Akt level to a 2.5-fold level compared to control group. And MPP⁺-induced down-regulation of Akt expressions was inhibited by SchiA treatment. These results indicated that SchiA could activate PI3K/Akt signaling pathway. To further validate whether the activation of PI3K/Akt signaling pathway, the inventors determined the blocking effect of PI3K/Akt inhibitor such as AKT inhibitor IV on the in vitro neuroprotective effect of SchiA. SH-SY5Y cells were pre-incubated with Akt inhibitor IV (2 μM) for 1 h and then treated with SchiA for 12 h. Drug-treated cells were further treated with 2 mM MPP for another 36 h, MTT assay was used to determinate cell viability. FIG. 16B showed that AKT inhibitor IV could clearly abrogate the protective effect of SchiA on MPP⁺-induced cytotoxicity. ###P <0.005 and ##p<0.05 versus control group. *p<0.05, **p<0.01 and *** p<0.005 versus MPP+ group. These findings suggesting PI3K/AKT signaling pathways as a critical role to regulate the cytoprotective effect of SchiA.

### Example 16

### Study on the Effect of SchiA in Increasing CREB Phosphorylation

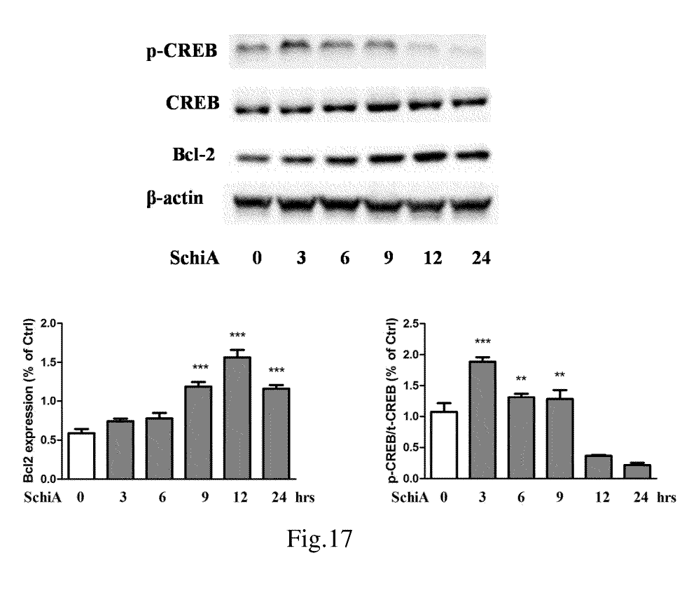

Since the inventors have demonstrated the neuroprotective effect SchiA is partially mediated by Bcl-2 activation. To investigate the effect of SchiA on the activation of the upstream targets of CREB in MPP⁺-treated SH-SY5Y cells, the protein levels of p-CREB/CREB and were determined. Cells were treated with 60 μM SchiA alone from 0 h to 24 h. Cells were lysed and examined by western blot. The results showed that SchiA could significantly stimulate phosphorylation of CREB after treatment for 3 h and was about 1.5-fold in a short term compared with 0 h (FIG. 17). Then p-CREB was decreased to the lower level, but Bcl-2 still was induced by SchiA from 3 h to 12 h. β-actin was shown as an internal control. **p<0.01 and *** p<0.005 versus control group. These findings suggest that CREB phosphorylation signal are increased at the beginning of treatment with SchiA and CREB/Bcl-2 signaling pathways as a critical role to regulate the expression by SchiA.

### Example 17

### Pharmacokinetic Study of SchiA

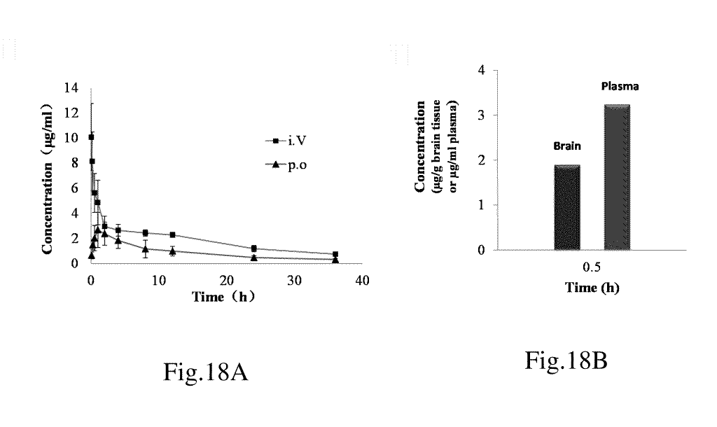

The concentration-time profiles of SchiA in rat plasma were presented in FIG. 18. And then non-compartmental model of DAS 2.0 program was chosen to calculate the major pharmacokinetic parameters. As shown in FIG. 18A, it expressed SchiA had two processes that contained rapid distribution and slowly excretion after administration. The main pharmacokinetic parameters, Tmax (1.33±0.58 h) was comparable to the results published by (Wei, Li et al. 2013). However, t1/2 (i.v: 15.88±1.82 h; p.o: 16.543±6.95 h) was longer than their results, which might be resulted from the rat species, drugs and dosage form. The absolute bioavailability of SchiA microemulsion was up to 47.32%. As shown in FIG. 18B, post-i.v bolus injection half an hour, the concentration of SchiA in brain which was equivalent of 54.5% of concentration in plasma.

### SUMMARY

The present study showed that SchiA is a potent neuroprotective agent in both SH-SY5Y cells in vitro and zebrafish in vivo. SchiA prevented 6-OHDA-induced DA neuron loss, rescued the deficit of locomotor behavior in zerbrafish. Furthermore, the inventors have shown that its neuroprotective activity might be exerted via the inhibition of NO overproduction by down-regulating the over-expression of iNOS in SH-SY5Y cells. SchiA also protects against the 6-OHDA induced SH-SY5Y cell death mediated by regulation of AKT/GSK3β expression. These findings demonstrate that SchiA may have potential therapeutic value for neurodegenerative disease associated with oxidative stress such as PD.

The present study also showed that SC possesses neuroprotective effect in MPP+-induced cell damage in SH-SY5Y cells, thus SC can be used to treat neurodegenerative disease caused by dopaminergic neuron loss, such as PD.

6-Hydroxydopamine (6-OHDA) is a selective catecholaminergic neurotoxin, has been widely used to investigate the pathogenesis and progression of PD. 6-OHDA is selectively taken up by the plasma membrane dopamine transporters and subsequently accumulates in the mitochondria (Saito, Nishio et al. 2007). It has been demonstrated that ROS plays an important role in cell apoptosis induced by 6-OHDA via auto-oxidation and generation of intracellular ROS (Fujita, Ogino et al. 2006). Therefore, initial blockage of ROS might be a very important factor for the protection of neurons. The results showed that the level of intracellular ROS significantly increased after SH-SY5Y cells were treated with 6-OHDA for 4 h. However, the level of intracellular ROS decreased in a dose-dependent manner when SH-SY5Y cells were pretreated with different concentrations of SchiA prior to 6-OHDA treatment. Moreover, NO, including iNOS and nNOS, is well known to be involved in the pathogenesis of PD (Kim, Kim et al. 2010). The data showed that SchiA reduced NO production and down-regulated the expression of iNOS induced by 6-OHDA. These results suggest that SchiA exerts a protective effect in the SH-SY5Y cells through anti-oxidative action and suppression of the iNOS-NO pathway.

The results depicted that 6-OHDA incubation for 4 h decreases GSK3β phosphorylation at serine 9 residue. However, pretreatment with SchiA completely blocked the repression of GSK-3β phosphorylation effect of induced by 6-OHDA. This result suggests the neuroprotective effect of SchiA and further shows the effect against 6-OHDA-induced cell death is partially mediated by GSK3β. Inactivation of GSK3β might attenuate neurodegeneration in PD and thus Schi A could serve as a potential agent for the therapy.

In addition, the PI3K-Akt and MAPK pathways play important roles in neuronal survival in both physiological and pathological states (Blum, Torch et al. 2001). A series of studies have well established that the PI3K-Akt and MAPK signaling pathways are survival and anti-apoptotic factor in multiple paradigms, including resistance against MPP+ neurotoxicity (Al-Nedawi, Meehan et al. 2008). In this study, the inventors found a decrease of phosphorylation of AKT and ERK after treatment with 6-OHDA, and pre-treatment with SchiA reversed the changes of the MAPK protein phosphorylation state induced by 6-OHDA. These results indicating that the PI3K-Akt and MAPK pathways play a crucial in the neuroprotective effects of SchiA in SH-SY5Y cells.

Moreover, the neuroprotective effect of SchiA against 6-OHDA induced neurodegeneration was confirmed in zebrafish model. 6-OHDA has been shown to dominate the DA neuronal death and injury of zebrafish brain and thus, has been demonstrated to be an appropriate model for PD (McKinley, Baranowski et al. 2005). Zebrafish have been reported previously as an easy in vivo model to screen native component for the neuroprotective effect in previous studies (Zhang, Cheang et al. 2011; Zhang, Cheang et al. 2012). In the present study, 6-OHDA exerted a significant impairment on zebrafish DA neurons and a reduction of locomotor behavior, which is consistent with earlier work (Anichtchik, Kaslin et al. 2004). Co-treatment of SchiA with 6-OHDA dramatically restored the DA neuron loss and recovered the locomotor movement reduction. This zebrafish in vivo data provides further confirmatory evidence supporting the observed neuroprotective effect of SchiA in vitro.

The anti-PD effect of the dibenzocyclooctadiene lignans and neuroprotective activity of SchiA or SC has never been reported. The inventors had systemmatically compare the major five dibenzocyclooctadiene lignans from the fruit of S. chinensis for both cytotoxicity and neuroprotective activity in vitro. Interesting, only SchiA and SC could protect SH-SY5Y cells against MPP⁺-induced cell death at comparable dosage range. To best of our knowledge, this is the first investigation to unravel the neuroprotective activity of SchiA and SC and the underlying mechanism for SchiA.

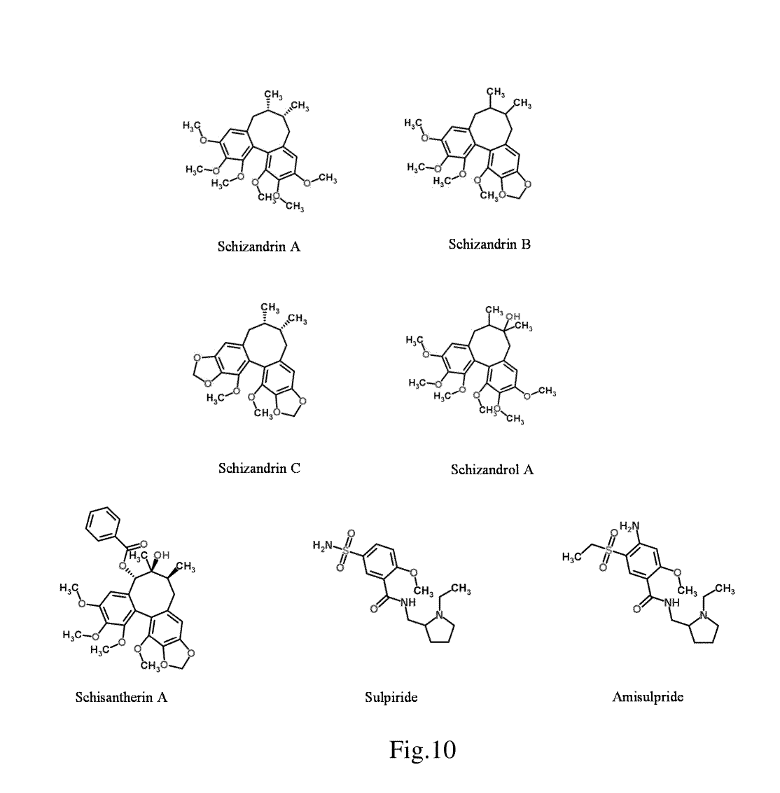

The inventors disclosed the relationship of the five lignans with dibenzocyclooctadiene skeleton (FIG. 10) and their neurprotective activity, which are SchizandrinA, SchizandrinB, SchizandrinC, schizandrolA and Schisantherin A, respectively.

Initially, two different dibenzocyclooctadiene lignans with methylenedioxy groups, SC and SchiA, exhibited neuroprotective effects in vitro, while no effects were observed on lignans without the methylenedioxy group (namely Schizandrin A and Schizandrol A). At the same time, comparing with SchizandrinB and SC, the increasing number of methylenedioxy group in dibenzocyclooctadiene skeleton enhanced the neuroprotective activity and decreased cytotoxicity. On the other hand, the number of methoxy groups in the benzene ring also affected the neuroprotective activity. In compound SC, SchiA and Schizandrol A, it showed that the less methoxy groups increased the neuroprotective effects. Comparing with the five dibenzocyclooctadiene lignans, compound SchiA showed the strongest neuroprotective activity. Therefore, the inventors deduce that:

(1) The C-8 and C-8′ positions of the cyclooctadiene ring are substituted with methylenedioxy groups in both schisandrin C and schisantherin A reveals methylenedioxy group(s) may be important substituent groups in dibenzocyclooctadiene lignans for their neuroprotective effects against MPP+ and MPTP induced toxicity; (2) methylenedioxy group(s) could not solely guarantee the activities, a large benzoyloxy substituent group on the cyclooctadiene ring in schisantherin A may improve the activities. Indeed, regarding to the structure of Schisantherin A and experimental result, the inventors believed the benzoyloxy and methoxyl group are both importance in treating neurodegenerative disease.

Since SchiA exhibited the strongest protective effect with the lower cytotoxicity among all tested lignans, the inventors investigated the possible molecular mechanisms of SchiA on MPP⁺-induced cytotoxicity in SH-SY5Y cells in vitro. The execution phase of apoptosis is generally activated by members of a distinct, highly conserved class of intracellular cysteine proteases, called caspases, characterized by their almost absolute specificity for aspartic acid residues in their substrates (Blum, Torch et al. 2001). Caspases plays an essential role in the death receptor apoptotic pathway and mitochondrial apoptotic pathway and the activation of caspase-3 has been shown to be involved in the apoptosis of DA neurons in PD (Hartmann, Hunot et al. 2000). Caspase-3 has been identified as an important protein in the final pathway of apoptosis. Caspase-3 activates DNA fragmentation factor, which in turn activates endonucleases to cleave nuclear DNA, and finally leads to apoptotic cell death. The results found that, SchiA suppressed MPP⁺-induced activation of caspase-3 and caspase-7, suggesting that SchiA may act upstream of caspase-3 to block apoptosis. Other study suggested that the decrease in caspase-3 activity correlates well with the decrease in the Bax/Bcl-2 ratio and it is in accordance (Cory and Adams 2002). However, whether the mechanisms by which SchiA inhibits MPP⁺-triggered activation of caspase-3 regulated by Bcl-2 family proteins, still needs to be confirmed.

Induction of cellular defense mechanism via AKT pathways by natural compound contributed to the cytoprotective effect had been reported as a great potential therapy in neurodegenerative diseases (Deng, Tao et al. 2012; Hsu, Chen et al. 2012). The PI3K-Akt pathways play important roles in neuronal survival in both physiological and pathological states (Blum, Torch et al. 2001) (Klein and Ackerman 2003). A series of studies have well established that the PI3K/Akt signaling pathway is a survival and anti-apoptotic factor in multiple paradigms, including resistance against MPP neurotoxicity (Al-Nedawi, Meehan et al. 2008). Some neurotrophic factors, including nerve growth factor and brain-derived neurotrophic factor, prevent neuronal cells from MPP⁺-induced apoptosis via activating the PI3-K/Akt pathway (Jourdi, Hamo et al. 2009). According to the western blot analysis of this invention, SchiA could activate phosphorylation of PI3K and Bcl-2 which are known to promote cell survival and to prevent apoptosis respectively. Based on the results described above, the inventors examined whether Akt and PI3K inhibitors could inhibit the cytoprotective effect of SchiA. The inventors found that the effect of SchiA was inhibited by AktIV, indicating that the PI3K/Akt pathway plays a crucial in the neuroprotective effects of SchiA in SH-SY5Y cells.

Bcl-2 family members are key regulators of apoptosis in general and of naturally occurring and pathological neuronal death in particular. Bcl-2 family members are involved in cell death processes caused by MPP⁺, with Bcl-2 being an anti-apoptotic protein while Bax exhibiting pro-apoptotic activity (O'Malley, Liu et al. 2003). Cell survival in the early phase of the apoptotic cascade depends mostly on the balance between the pro- and anti-apoptotic proteins of the Bcl-2 family, hereof, the Bax/Bcl-2 ratio may better predict the apoptotic fate of the cell than the absolute concentrations of either protein alone (Tanaka, Asanuma et al. 2004). The results of this invention shows that SchiA pre-treatment significantly increases the expression of anti-apoptotic Bcl-2 and has no impact on the expression of pro-apoptotic Bax. Thus, the effect of SchiA on MPP⁺-induced apoptosis may be, at least partly, mediated by regulating the expression of Bax and Bcl-2. These results suggested a notion that SchiA treatment shifted the balance between positive and negative regulators of apoptosis towards cell survival. Many Studies showed that there are extensive cross-talk occurs between CREB, PKA, and mitogen-activated protein kinase (MAPK) pathway (Lonze and Ginty 2002). In particular, Bcl-2 upregulation require CREB activation (Finkbeiner 2000). Thus, the results suggest that the CREB activated upregulation of Bcl-2 may be one mechanism underlying the neuroprotective effect of SchiA.

In this present study, the inventors demonstrated the neuroprotective effects of SchiA in anti-apoptosis as well as cell survival signaling pathways. The findings provide the documentation of protective effects of SchiA against MPP⁺/MPTP-induced cytotoxicity and neurotoxicity in vivo and in vitro. In this neuroprotection, SchiA significantly inhibited MPP⁺-induced cytotoxicity, prevented caspase-3 activation, and increase Bcl-2 expression. Moreover, the inventors found that SchiA modulates the activation of the Akt/CREB/Bcl-2 cell survival signaling pathway.

The in vitro study showed SchiA was found to reduce MPTP-induced TH-positive dopermaneric neuron loss and/or locomotion deficit in PD zebrafish and mice models. The concentration-time profiles of SchiA in rat plasma were also studied (supplementary information). It expressed SchiA had two processes of rapid distribution and slowly excretion after administration. The inventors also found SchiA absorbed quickly and eliminated slowly in plasma, maintain the higher plasma concentration, demonstrating good behavior in vivo from a pharmacokinetic perspective.

The treatment of brain disorders drugs should pass through the blood-brain barrier (BBB) to regulate certain receptors in the CNS for producing effect. Thus the inventors examined whether SchiA could pass BBB. The results showed that SchiA is able to traverse the BBB in vivo. But as time went on, the concentration of Schi A in brain was dramatically reduced and wasn't detected in brain samples up to 6 h. Moreover, it wasn't detected in brain under oral administration, however the dose was increased and the plasma concentration was considerable. Generally, drug distribution was depends on the free drug concentration in plasma which could affect the supply to the target disuse. It has been demonstrated that SchiA was a potent P-gp inhibitor (Pan, Lu et al. 2006). P-gp, functioning as an ATP-dependent drug pump, efficiently extrudes intracellular drugs out of cells, especially anticancer drugs. After oral administrated to plasma, lots of SchiA were combined at intestines and hardly entered into brain by passing through the barrier of P-gp. But after an i.v. bolus injection, SchiA which liked an impact was directly entered into systemic circulation and P-gp was saturated with SchiA speedily and SchiA couldn't stay at brain as long as plasma despite the concentration was considerable. Therefore, it is rationalized the SchiA passed through BBB caused by i.v. and it has been hypothesized that P-gp is the major determinant of SchiA pharmacokinetics.

The Pharmacokinetic study indicating the pharmaceutical preparation dosage form of SchiA can be modified to significantly enhanced oral bioavailability. Nanoparticles of Schi A was supposed to improve the solubilization of the surfactant, the noncrystalline state of the drug in the matrix and the fast dissolution rate compared to pure drug suspension (Pei, Lv et al. 2013).

In conclusion, the results from the in vitro and in vivo assays in this study demonstrate the neuroprotective effects of SchiA, and add insight into its mechanism of action in 6-OHDA-induced or MPP⁺/MPTP-induced cytotoxicity and neurotoxicity. The data indicates that pretreatment with SchiA is able to regulate intracellular ROS level to activate AKT/GSK3β pathways against 6-OHDA-induced oxidative damage. Also, the in vivo zebrafish data reported here demonstrate that SchiA can prevent DA neuron loss induced by 6-OHDA. This study also shows the neuroprotective effect of SC. Thus, the findings further indicate that SchiA and SC can be both candidates for the treatment and/or prevention of neurodegenerative diseases such as PD.

What is claimed is:

1. A method of treating and/or preventing neurodegenerative disease comprising administrating a pharmaceutically effective amount of a compound to a subject in need thereof, wherein said compound is Schisantherin A, Schizandrin C or a combination thereof.

2. The method of claim 1, wherein said neurodegenerative disease is caused by dopaminergic neuron loss or Aβ1-42 induced memory impairment.

3. The method of claim 1, wherein said neurodegenerative disease is selected from the group consisting of Parkinson's disease and Alzheimer's disease.

4. A pharmaceutical composition comprising a pharmaceutically effective amount of a compound and a pharmaceutically acceptable carrier, wherein said compound is Schisantherin A, Schizandrin C or a combination thereof.

5. The pharmaceutical composition of claim 4, wherein said composition can be prepared as a formulation selected from the group consisting of microemulsion, tablets, granules, injection, powder, solution, suspension, sprays, patches and capsules.

6. A method of treating and/or preventing neurodegenerative disease comprising administrating said pharmaceutical composition of claim 4 to a subject in need thereof.

7. The method of claim 6, wherein said neurodegenerative disease is caused by dopaminergic neuron loss or Aβ1-42 induced memory impairment.

8. The method of claim 6, wherein said neurodegenerative disease is selected from the group consisting of Parkinson's disease and Alzheimer's disease.
